(19) United States
(12) Patent Application Publication
(10) Pub. No.: US 2014/0194840 A1
(43) Pub. Date: Jul. 10, 2014

(54) PORTABLE AUTOMATED BODY FLUID DRAIN CONTROL APPARATUS

(71) Applicant: BeckerSmith Medical, Inc., Los Angeles, CA (US)

(72) Inventor: Jan M. Eckermann, Los Angeles, CA (US)

(73) Assignee: BeckerSmith Medical, Inc., Los Angeles, CA (US)

(21) Appl. No.: 14/210,224

(22) Filed: Mar. 13, 2014

Related U.S. Application Data
(63) Continuation-in-part of application No. 13/931,648, filed on Jun. 28, 2013.
Application No. 13/931,648 is a division of application No. 12/197,201, filed on Aug. 22, 2008, now Pat. No. 8,475,419.
(60) Provisional application No. 61/779,751, filed on Mar. 13, 2013.
(60) Provisional application No. 60/966,132, filed on Aug. 25, 2007.

(51) Int. Cl. A61M 27/00; A61M 1/00

(52) U.S. Cl. CPC A61M 27/00; A61M 1/0019
USPC 604/328

(57) ABSTRACT

A portable body fluid collection device that includes an apparatus for controlling the collection of body fluids and at least one strap associated with the apparatus for controlling the collection of body fluids. The strap can be worn by a patient to support the weight of the automated body fluid drain control apparatus. The apparatus for controlling the collection of body fluids includes a drainage tube and a fluid collection chamber in fluid communication with the drainage tube. The fluid collection chamber includes a first valve, and the fluid collection chamber is configured such that when a predetermined amount of fluid is collected in the collection chamber before a first predetermined period of time elapses, the collection chamber ceases collecting fluid.

### CROSS REFERENCE TO RELATED APPLICATIONS

This application claims the benefit of U.S. Provisional Application No. 61/779,751 filed Mar. 13, 2013, which is incorporated herein by reference in its entirety. This application is also a continuation-in-part of U.S. application Ser. No. 13/931,648 filed Jun. 28, 2013, which is a divisional application of U.S. patent application Ser. No. 12/197,201, filed Aug. 22, 2008, now U.S. Pat. No. 8,475,419, which claims the benefit of U.S. Provisional Application No. 60/966,132 filed Aug. 25, 2007, the entireties of which are all incorporated by reference herein.

### FIELD OF THE INVENTION

The present invention relates to a portable body fluid drain control apparatus.

### BACKGROUND OF THE INVENTION

Body fluid drains and containers are well known in the art. For example, there are collection devices for urine and others that drain and collect spinal fluid. None of these devices are able to easily control the drainage rate of the fluid as a function of time.

In connection with the drainage of cerebrospinal fluid (“CSF”), for most people, the body produces 450 ccs of CSF over a 24 hour period which fills the subarachnoid space in the body. There are many instances where it may be advisable and/or necessary for some of the CSF to be drained. For example, during certain medical procedures such as brain surgery, the surgeon may wish to drain some of the CSF in order to retract the brain. In addition, in some brain and spinal surgeries where the dura mater is penetrated, the CSF would need to be partially drained to keep pressure off the wound site in order to allow it to heal. Also, in certain head trauma cases where CSF is collecting in the cranial cavity, it may be preferable to drain some of the CSF from the subarachnoid space in the lumbar spinal region to relieve the pressure on the brain.

Conventional methods of draining CSF involve tapping into the cranial or subarachnoid space in the spinal column and draining the excess CSF through a catheter tube into a collection bag. The amount of drainage must be regulated, as if there is too much drainage, a patient can be irreversibly injured or can be fatally injured.

Unfortunately, the rate at which the CSF drains is not linear in fashion. For example, the CSF can drain at 1 cc per hour and then suddenly drain 5 ccs in 10 minutes. Since there are irreversible and potentially fatal consequences if too much CSF is drained, the volume of the drainage has to be constantly monitored by a nurse. Due to the demand on a nurse's time and the non-linearity of the drainage, there is a potentially fatal margin of error. Thus, an apparatus that continuously monitors and controls the drainage of the CSF would be of great benefit to the art.

Moreover, patients having CSF fluid or other fluids drained are often bed ridden due to the size of the monitoring machines. Accordingly, the ability for a patient to be able to get up out of bed and move around while the fluid is still being drained is advantageous.

### SUMMARY OF THE PREFERRED EMBODIMENTS

In accordance with a first aspect of the present invention there is provided a portable body fluid collection device that includes an apparatus for controlling the collection of body fluids and at least one strap associated with the apparatus for controlling the collection of body fluids. The strap can be worn by a patient to support the weight of the automated body fluid drain control apparatus. The apparatus for controlling the collection of body fluids includes a drainage tube and a fluid collection chamber in fluid communication with the drainage tube. The fluid collection chamber includes a first valve, and the fluid collection chamber is configured such that when a predetermined amount of fluid is collected in the collection chamber before a first predetermined period of time elapses, the collection chamber ceases collecting fluid. In a preferred embodiment, the apparatus for controlling the collection of body fluids further includes a measuring device that measures the amount of fluid entering the collection chamber through the first valve during the predetermined first period of time. The first valve has an open and a closed state. The first valve is normally open and allows body fluids to flow into the collection chamber. During the first period of time, if a predetermined volume of body fluid enters the chamber, the first valve is closed.

In a preferred embodiment, the apparatus for controlling the collection of body fluids further includes a second valve having an open and a closed state. The second valve is normally closed so that body fluids collect in the chamber, and the second valve can be opened to empty the collection chamber. Preferably, after the first period of time has elapsed, the first valve is opened and the second valve is closed and a second period of time starts. In this embodiment, the first period of time is equal to the second period of time. In a preferred embodiment, the portable body fluid collection device further includes a container. The apparatus for controlling the collection of body fluids is disposed in the container, and the strap is connected to the container.

In a preferred embodiment, the drainage tube is configured to be inserted into a patient's subarachnoid region at an insertion point. The container and strap are configured such that when the container is worn by a patient, the first valve is positioned below the insertion point. Preferably, the container is vertically adjustable between at least a first and a second position. Preferably, the container is a backpack.

In accordance with another aspect of the present invention there is provided a method of collecting body fluid that includes providing an apparatus for controlling the collection of body fluids, inserting an end of the drainage tube into a patient's subarachnoid region at an insertion point, and securing the apparatus for controlling the collection of body fluids to the patient's body at a first position, wherein in the first position, the first valve is located vertically below the insertion point. The apparatus for controlling the collection of body fluids includes a drainage tube, and a fluid collection chamber in fluid communication with the drainage tube. The fluid collection chamber includes a first valve, and the fluid collection chamber is configured such that when a predetermined amount of fluid is collected in the collection chamber before a first predetermined period of time elapses the collection chamber ceases collecting fluid. Preferably, the apparatus for controlling the collection of body fluids is secured to the patient's body via a strap. In a preferred embodiment, the strap is connected to a container, and at least a portion of the apparatus for controlling the collection of body fluids is disposed in the container.

In a preferred embodiment of the present invention, an automated fluid collection apparatus comprises a first tube having a first end connected to a drain that has been inserted into a patient's subarachnoid region and a second end connected to an opening at the proximal end of a first collection chamber. The first collection chamber also has an opening at the distal end thereof. In one embodiment the chamber has a first valve or first controllable closing mechanism proximate the opening at the proximal end and a second valve or controllable closing mechanism at the distal opening thereof. In another embodiment, there is a second tube having a first end and a second end, whereby the first end is connected to the opening at the distal end of the collection chamber. In the preferred embodiments of the present invention, the first and second valves may be located on the first and second tubes respectively or on the proximal and distal ends of the first chamber. In one preferred embodiment, the distal end is connected directly to a collection bag; in another embodiment the second tube is connected at the second end to a second collection chamber or bag.

In the preferred embodiment, the apparatus also comprises a measuring device which determines the amount of fluid that is being collected in the first chamber over a preselected period of time. In one preferred embodiment, the measuring device is an optical sensor connected to a spectrophotometer which is able to detect specific wavelengths inside the chamber so as to determine the amount of fluid collected herein. In other alternate embodiments, a different type of fluid measuring device may be used. In an alternate embodiment, the measuring device may also be able to sense the type of fluid collected in the chamber to detect any anomalies therein.

The apparatus also comprises a timer, microprocessor and power supply which are connected to the measuring device and to the controllable closing mechanisms so that the amount of fluid collected in the first collection chamber is constantly being measured, monitored and controlled. The microprocessor processes the various measurements received from the measuring device to determine the volume contained within the first chamber. The timer controls the period of time over which the fluid is measured and collected within the first chamber and is reset for each new period of time as instructed by the processor. Once the microprocessor determines that the first chamber is filled to a preselected level at any time prior to the expiration of the selected time period, it will cause the first valve to close and the second valve to open such that the first chamber will be emptied and so that the drainage will discontinue for the remainder of the preselected time period. In this manner, the drainage rate of the fluid will never be more than the preselected level during the preselected period of time.

In the preferred method of the present invention, the first end of the first tube is attached to a drain that has been inserted into the appropriate subarachnoid region of the body. The first valve is opened and the second valve is closed. The timer is also set for either a default or an alternate timer period as is the maximum volume for the first chamber.

Thereafter, the CSF fluid drains through the proximal valve into the first collection chamber through the use of gravity. As the fluid drains into the first chamber, the volume of the first chamber is constantly measured by the measuring device. Once the microprocessor determines that the first chamber is filled to a preselected level at any time prior to the expiration of the then applicable preselected time period, it will cause the first valve to close and the second valve to open such that the first chamber will be emptied and so that the drainage will discontinue for the remainder of the preselected time period. In this manner, the drainage rate of the fluid will never be more than the preselected level during the preselected period of time. At the end of each preselected time period the timer will reset.

If the first chamber has not attained the maximum preselected volume in the preselected time period, the first valve will remain open and the second valve will remain closed and the drainage will continue until the maximum volume is attained.

If at any time there is any problem with the system or if the first collection chamber fails to fully empty at each predetermined interval, an alarm will notify the appropriate personnel that their attention is required.

The invention, together with additional features and advantages thereof, may be best understood by reference to the following description.

### BRIEF DESCRIPTION OF THE DRAWINGS

FIG. 1 is a perspective view of a preferred embodiment of the apparatus of the present invention in a closed position;

FIG. 2 is a partial view of an alternate e embodiment of the apparatus of the invention in a closed position;

FIG. 3 is an exploded view of the preferred embodiment of FIG. 1 in an open position;

FIG. 4 is a perspective view of a portable version of the apparatus of the present invention that can be rolled;

FIG. 5 is a perspective view of a portable body fluid collection device in accordance with a preferred embodiment of the present invention;

FIG. 6 is a perspective view of the portable body fluid collection device of FIG. 5 adjusted to a lower position;

FIG. 7 is a perspective view of another portable body fluid collection device in accordance with a preferred embodiment of the present invention; and

FIG. 8 is a perspective view of another portable body fluid collection device in accordance with a preferred embodiment of the present invention.

Like numerals refer to like parts throughout the several views of the drawings.

### DETAILED DESCRIPTION OF THE PREFERRED EMBODIMENTS

The following description and drawings are illustrative and are not to be construed as limiting. Numerous specific details are described to provide a thorough understanding of the disclosure. However, in certain instances, well-known or conventional details are not described in order to avoid obscuring the description. References to one or an other embodiment in the present disclosure can be, but not necessarily are, references to the same embodiment; and, such references mean at least one of the embodiments.

Reference in this specification to “one embodiment” or “an embodiment” means that a particular feature, structure, or characteristic described in connection with the embodiment is included in at least one embodiment of the disclosure. Appearances of the phrase “in one embodiment” in various places in the specification do not necessarily refer to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks: The use of highlighting has no influence on the scope and meaning of a term; the scope and meaning of a term is the same, in the same context, whether or not it is highlighted. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

It will be appreciated that terms such as “front,” “back,” “top,” “bottom,” “side,” “short,” “long,” “up,” “down,” and “below” used herein are merely for ease of description and refer to the orientation of the components as shown in the figures. It should be understood that any orientation of the components described herein is within the scope of the present invention.

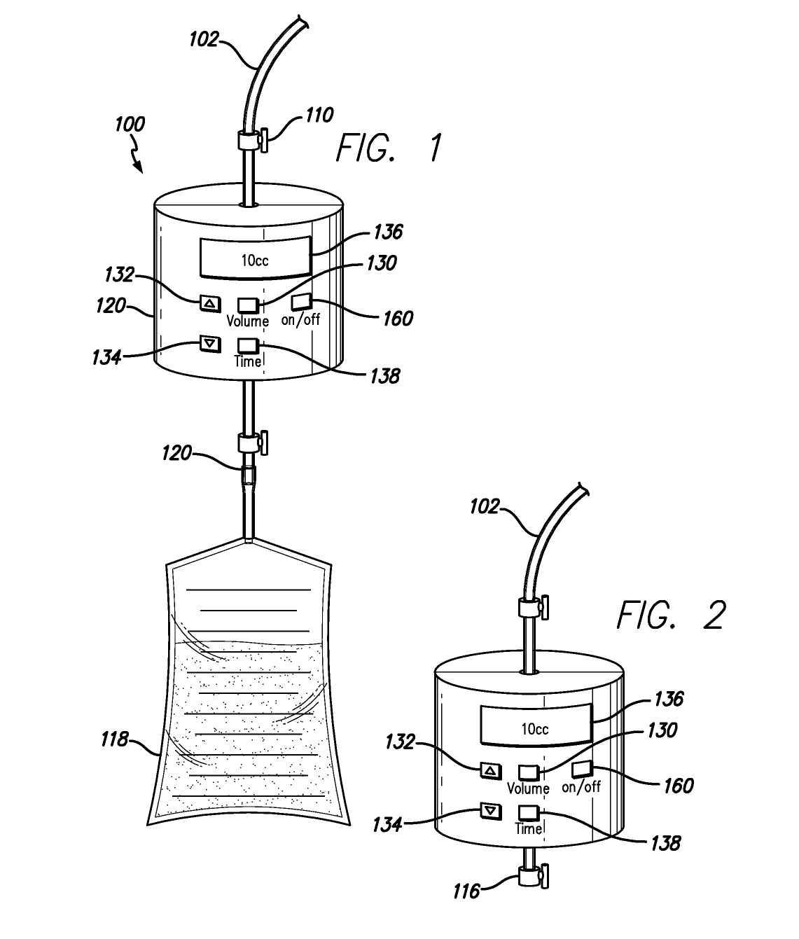

Generally, the present invention may be briefly described as follows. Referring first to FIGS. 1 and 2, a preferred embodiment of an automated fluid drain control apparatus 100 of the present invention is shown.

The automated fluid drain control apparatus 100 comprises a comprises a first tube 102 having a first end (not shown) connected to a drain (not shown) that has been inserted into a patient's subarachnoid region and a second end 104 connected to an opening 106 at the proximal end of a first collection chamber 108. In a preferred embodiment, the first collection chamber 108 has a first valve 110 or other suitable first controllable closing mechanism known in the art that is capable of opening and closing the flow of fluid between first tube 102 and the first collection chamber 108. In the preferred embodiments shown in FIGS. 1, 2 and 3, valve 110 is located on tube 102. However, in an alternate embodiment, not shown, there is no valve 110. In the embodiment shown, valve 110 is a manual valve that can be opened and closed by the user of the apparatus. However, in alternate embodiments, the valve 110 can be controlled electronically.

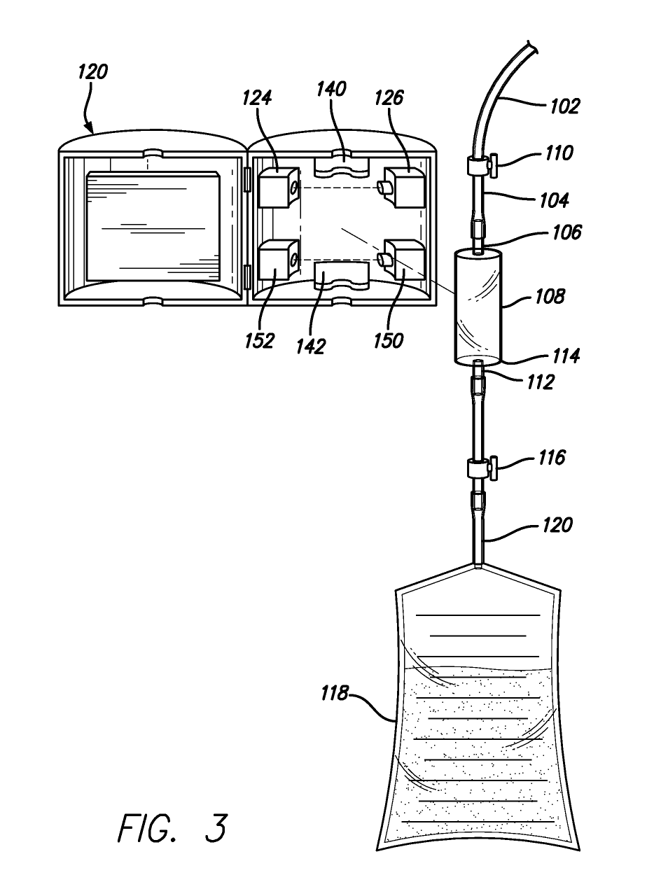

The first collection chamber 108 also has an opening 112 at the distal end 114 thereof. In FIG. 2, an alternate embodiment is shown in which the first collection chamber 108 has a second valve 116 or other suitable second controllable closing mechanism known in the art that is capable of opening and closing the flow of fluid out of the distal end 114 of the first collection chamber 108. In the embodiments shown in FIGS. 1 and 3, there is a second collection chamber 118 with a neck 120 which is connected to the opening 112 at the distal end 114 of the first collection chamber 108. In yet another embodiment (not shown) a second tube having a first end and a second end is connected at the first end to the opening 112 at the distal end 114 of the collection chamber 108. In that embodiment, the second tube is also connected at the second end to a second collection chamber or bag such as the one shown in FIGS. 1 and 3.

In a preferred embodiment, the apparatus 100 also includes a housing 120 which houses the first chamber 108 and the electronics (not shown) which include a microprocessor (not shown), a timer (not shown), a power supply (not shown) and relays (not shown) which control third and fourth valves 140 and 142. The third 140 and fourth 142 valves are either pinch valves or any other mechanism known in the art that are controllable and which can easily and quickly impede the flow of fluid. In addition, in a preferred embodiment shown in FIG. 3, the third and fourth valves 140 and 142 are located in the housing proximate the beginning of the first and second tubes so that they can impede the flow of fluid into and out of the first collection chamber 108. However, in alternate embodiments there is only one set of valves such that either valves 102 and 116 are controlled electronically such that there is no need for third and fourth valves or valves 102 and 116 are unnecessary. However, in order to provide a fail safe mechanism, in the preferred embodiment, all four valves are used.

In addition, the housing 120 also contains a first measuring device connected to the microprocessor which together determine the amount of fluid that is being collected in the first chamber 108 over a preselected period of time. In one preferred embodiment, shown in FIG. 3 the measuring device is an optical sensor 124 connected to a spectrophotometer 126 which is able to detect specific wavelengths by shining a light through the first collection chamber 108 so as to determine the amount of fluid collected therein. In other alternate embodiments, a different type of fluid measuring device may be used with suitable modifications to the electronics and microprocessor. By way of example, and not limitation, such measuring devices may include, a weight measuring device that measures the amount of fluid collected within the first collection chamber 108 based upon weight; an acoustic sensor coupled with a sound generator which sends sound waves through the chamber 108 in order to detect the amount of fluid based upon the change in the sound waves as they traverse the fluid; a capacitive sensor either located within the first collection chamber to measure a variable such, as, but not limited to, the pressure inside the chamber created by the changes in the volume of fluid to determine the volume or depending on the material of the chamber, detect and measure deformations of the chamber again to determine volume; a flowmeter; a thermo sensor; a ph sensor; a device that uses the technology of microfluidics; or any other fluid measuring device known in the art.

In various embodiments of the present invention with suitable additional software added to the microprocessor, the measuring device also is able to detect any anomalies such as, but not limited to, the presence of blood, white cells, pus, dye, etc.

The microprocessor is connected to the measuring device, the timer and the relays. In the embodiment shown in FIGS. 1-3, the relays are connected to the third and fourth valves 140 and 142. However, in an alternate embodiment in which there are no third and fourth valves, the relays are connected to the first and second valves 110 and 116.

The microprocessor ensures that the fluid inside the first collection chamber constantly is being measured, monitored and controlled. The microprocessor also processes the various measurements received from the measuring device to determine the volume contained within the first chamber.

In one embodiment, the user can select the maximum volume that can be collected in the first collection chamber 108 during a selected period of time. In the embodiment shown in FIGS. 1 and 2, that is accomplished by pressing the volume control button 130 located on the front of housing 120 and thereafter pressing up and down indicator buttons 132 and 134 which sends a signal to the microprocessor which then stores the amount of maximum volume selected in its memory. Alternate selections means well known in the art can be used in lieu of the buttons shown in FIGS. 1 and 2. In addition, in embodiments such as is shown in FIGS. 1 and 2, an visual display 134 is shown on the front of the housing 120 which shows the volume amount selected.

In another embodiment, the microprocessor selects the default value for the amount of volume that can be selected during a selected period of time. Regardless of how the volume is selected, the volume value regulates how many times the chamber will empty per preselected time period.

In another embodiment, the user can select the period of time over which the fluid is measured and collected within the first chamber. In the embodiment shown in FIGS. 1 and 2, that is accomplished by pressing the timer control button 138 located on the front of housing 120 and then up and down indicator buttons 132 and 134 which sends a signal to the microprocessor which then stores the amount of time selected in its memory. In addition, in an embodiment such as is shown in FIG. 1, the visual display 136 is shown on the front of the housing 120 which shows the timer amount selected. This also will regulate how many times the chamber will empty per preselected time period. If the timer is not manually set by the user, then the timer will set a default for the preselected time period. In another embodiment, which does not have a manual controller, the timer may be preselected by the microprocessor. In yet a further embodiment, the apparatus provides either means of selecting the period of time.

Regardless of the embodiment used, once the microprocessor determines that the first chamber is filled to a preselected level at any time prior to the expiration of the then applicable preselected time period, it will cause the appropriate valve to close which in the preferred embodiment shown in FIG. 3 is third valve 140 and the appropriate other valve to open which in the preferred embodiment shown in FIG. 3 is the fourth valve 142. In alternative embodiments in which only valves 110 and 116 are being used, then the microprocessor will cause first valve 110 to close and second valve 116 to open. In this manner, the first collection chamber 108 will be emptied and drainage will discontinue for the remainder of the preselected time period. As a result, the drainage rate of the fluid will never be more than the preselected level during the preselected period of time.

In addition, in various embodiments, the microprocessor also can determine and display the total volume drained over a larger time period on display 136 by holding down the volume button for a preselected period of time, although other embodiments may use other techniques well known in the art for obtaining the information with suitable modifications of the electronics.

In addition, to the foregoing, the apparatus may also contain an additional spectrophotometer 150 and related sensor 152 to assure the complete emptying of the first collection chamber at the end of each appropriate cycle. In addition, the apparatus may also contain an alarm producing mechanism (not shown) whereby if the information sent by the measuring device as processed by the microprocessor detects that blood or other types of fluid or cells are present in the fluid, the nursing staff will be alerted. Likewise, with suitable additional electronics, other alerts may be present such as when there is a kink in the tubing, the power fails, or one of the components of the system is not operating properly, or if the first chamber fails to completely empty as required.

In the preferred method of the present invention, the first end of the first tube 102 is attached to a drain (not shown) that has been inserted into the appropriate subarachnoid region of the body. The appropriate valves associated with the proximal opening 106 of the first collection chamber 108 are opened and the appropriate valves associated with the distal end 112 of the collection chamber 108 are closed. In embodiments using four valves such as those shown in FIG. 3, the first and third valves 110 and 140 are opened and the second and fourth valves 116 and 142 are closed. The timer is also set for either a default or an alternate timer period and the maximum volume for the first chamber is also selected.

Thereafter, the fluid drains through the proximal valve(s) into the first collection chamber 108 through the use of gravity. As the fluid drains into the first chamber 108, the volume of the first chamber is constantly measured by the measuring device. Once the microprocessor determines that the first chamber 108 is filled to a preselected volume at any time prior to the expiration of the then applicable preselected time period, it will cause the proximal valve(s) to close and the distal valve(s) to open such that the first chamber will be emptied and the drainage will discontinue for the remainder of the preselected time period. In this manner, the drainage rate of the fluid will never be more than the preselected level during the preselected period of time. At the end of each preselected time period the timer will reset.

If the first chamber has not attained the maximum preselected volume in the preselected time period, the proximal valve(s) will remain open and the distal valve(s) will remain closed and the drainage will continue until the maximum volume is attained.

If at any time there is any problem with the system or if the first collection chamber fails to fully empty at each predetermined interval, an alarm will notify the appropriate personnel that their attention is required.

In addition, in the embodiments shown in FIGS. 1 and 2, the housing has an on/off button 160. Further, in the preferred embodiments, the tubing, bags, collection chamber and first and second valves are all disposable and replaceable. Those skilled in the art will understand that this type of apparatus is designed for use with CSF but it can be used in any other application in which the drainage amount of fluids over time is critical.

In another embodiment, the automated fluid drain control apparatus 100 includes a redundant fluid measuring system.

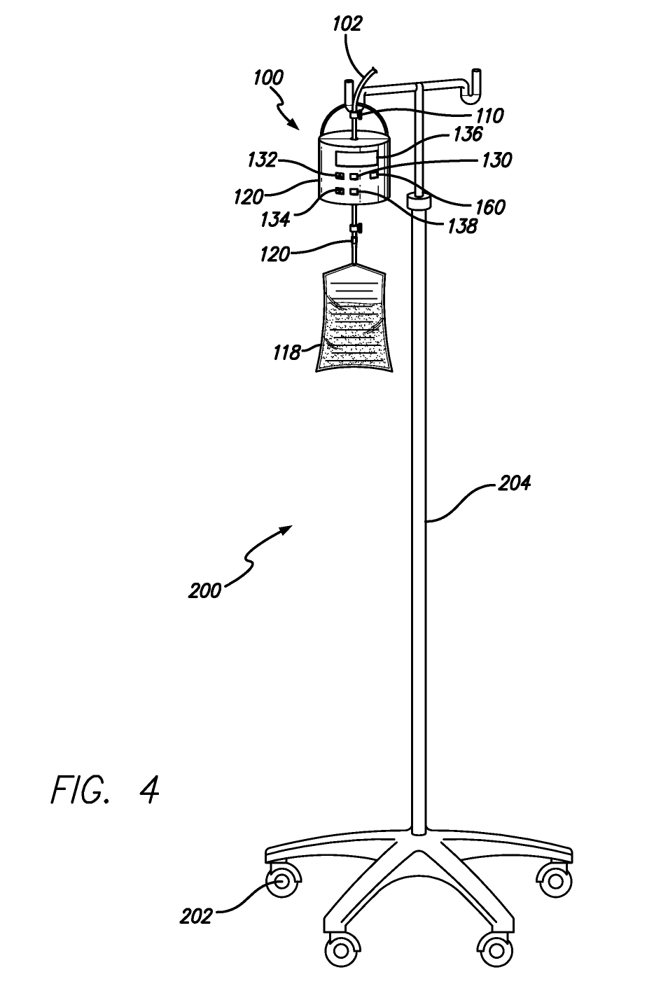

As shown in FIGS. 4-8, in other embodiments, the automated fluid drain control apparatus 100 is portable. As described above, in the prior art, close monitoring of patients having CSF drained is necessary. This confined a patient to bed. However, with the present automated system, a patient does not have to be as closely monitored by hospital personnel. Accordingly, the automated fluid drain control apparatus can be portable. For example, as shown in FIG. 4, in an embodiment of the invention, the automated fluid drain control apparatus 100 can be part of a tower assembly 200 that includes wheels 202. The apparatus 100 can be mounted on a tower 204 via a strap or permanently affixed thereto.

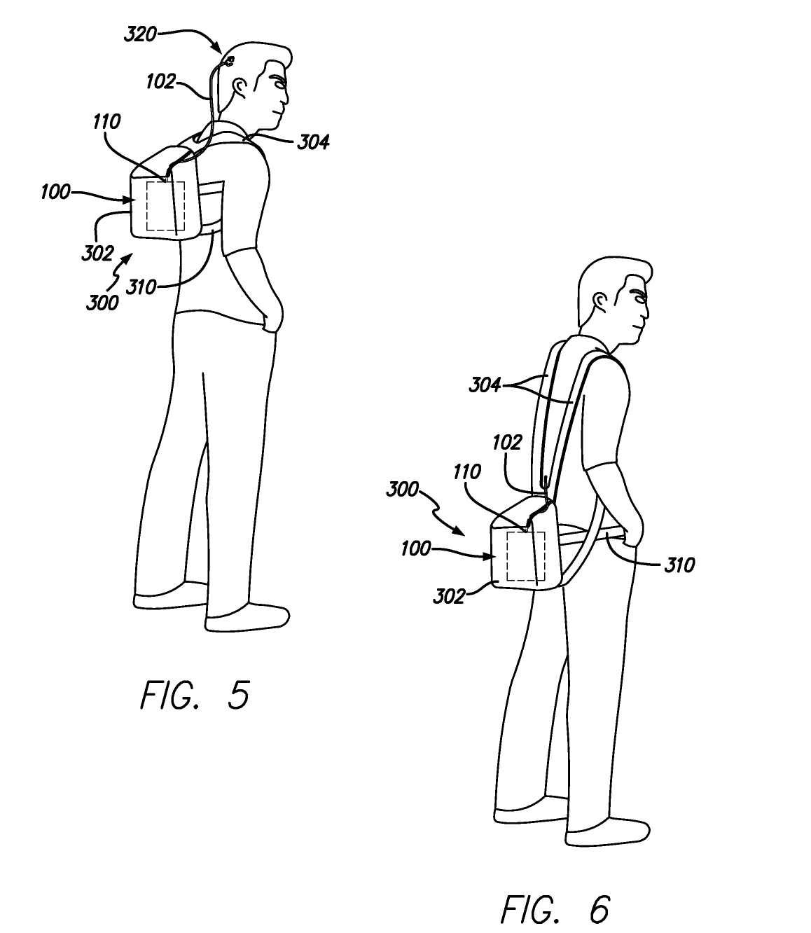

FIGS. 5-6 show a preferred embodiment of a portable body fluid collection device 300, that includes an apparatus for controlling the collection of body fluids 100, similar to that described above, drainage tube 102 and a container 302 for supporting or carrying the apparatus 100. As shown in FIGS. 5-6, in a preferred embodiment, the container 302 includes a first set of straps 304 that can be worn over the patient's shoulders, similar to a backpack. Preferably, the straps 304 are adjustable. In a preferred embodiment, the device also includes a second set of straps 310 that are worn around the front of the patient and clipped, clasped, tied or otherwise secured together. Preferably, straps 310 are also adjustable. It will be appreciated that the container 302 can be any device for carrying or supporting the apparatus 100. For example, the container 302 can be a bag, sack, box, shelf, purse, etc. In another preferred embodiment, the container 302 is a jacket into which the apparatus 100 is incorporated. Any type of wearable garment or container that can support the weight of the apparatus 100 and allow the tube 102 to extend into the subarachnoid space is within the scope of the present invention. For example, see the backpack taught in U.S. Publication No. 2012/0085804, published Apr. 12, 2012, the entirety of which is incorporated by reference.

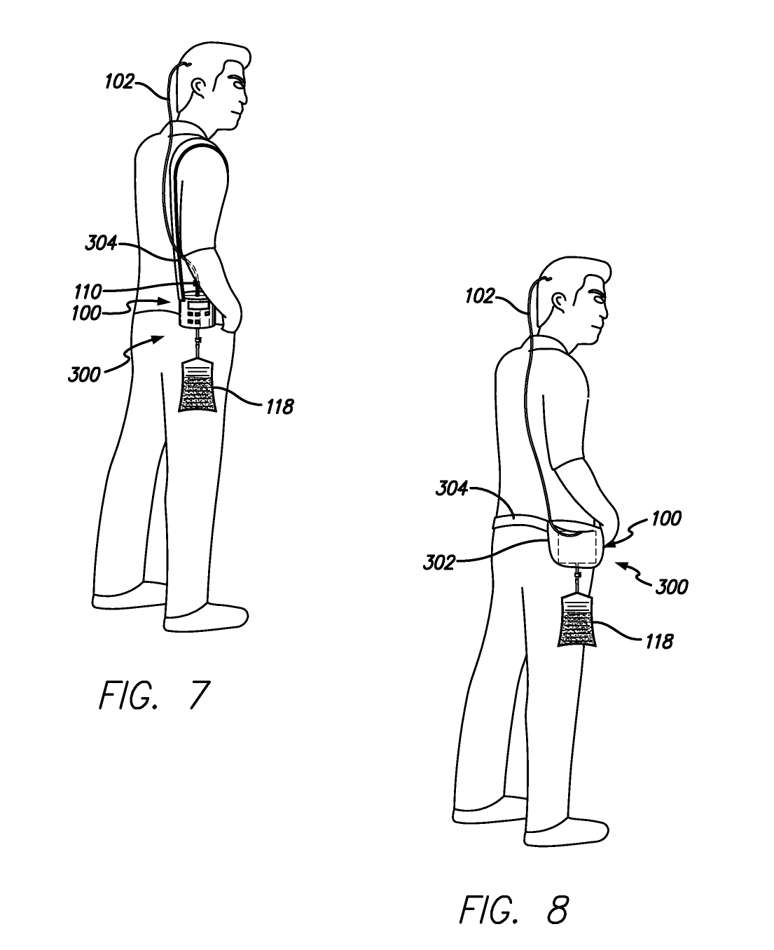

As shown in FIG. 7, in another embodiment, one or more straps 304 can be connected directly to the apparatus 100 and the container 302 can be omitted. In this embodiment, the strap(s) 304 can be worn by a patient to support the weight of the apparatus 100. For example, as shown in FIG. 7, the strap 304 can be worn like a purse strap so that the device 100 hangs near the patient's side.

In use, the drainage tube 102 (which may extend through an opening in the container) is inserted into the patient's subarachnoid region at an insertion point. The container 302, straps 304 and apparatus 100 are configured and positioned such that the first valve 110 is positioned below the insertion point, thereby allowing fluid to drain into the collection chamber via gravity. In a preferred embodiment, one or both of the container 302 or the straps 304 are adjustable in at least a vertical direction so that the first valve 110 can always be positioned below a chosen insertion point. FIG. 5 shows the positioning of the adjustable height container 302 when the tube 302 is inserted into an insertion point in the patient's skull. FIG. 6 shows the positioning of the adjustable height container 302 when the tube 302 is inserted into an insertion point in the patient's lower spine.

As shown in FIG. 8, in another embodiment, the container 302 and strap 304 can be formed as a fanny or hip pack. In this embodiment, the container 302 can be positioned on the patient's side making the device 100 and the first 108 or second collection chamber 118 more accessible. In the embodiment shown in FIG. 8, the second collection chamber 118 or collection bag is located outside the container 302 so that fluid can be drained more easily. Therefore, the container 302 can have an opening (e.g., with a zipper, buttons, Velcro, etc.) that the container (and any associated tubing) extends through. However, in another embodiment, the collection bag can be located in the container 302. Similarly, the back pack embodiment above can include an opening therein that allows the collection bag to hang outside the container.

As shown in FIG. 5, in another embodiment, the portable body fluid collection device 300 can be combined with a device 320 that signals to a user when the tube 102 is being pulled. Co-pending and simultaneously filed patent application Ser. No. 14/209,983, filed Mar. 13, 2014 and titled Fluid Drain Tube with Connector (attorney docket no. 69638-5010), the entirety of which is incorporated by reference herein, describes a device 320 that pulls on a user's hair or skin when the tube 102 is pulled in. Device 320 can be combined with portable body fluid collection device 300 and sold as a kit. In this manner, with a person wearing portable body fluid collection device 300, when they are moving around, device 320 will signal to the person that the tube 102 is moving and may help prevent withdrawal of the tube.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description of the Preferred Embodiments using the singular or plural number may also include the plural or singular number respectively. The word “or” in reference to a list of two or more items, covers all of the following interpretations of the word: any of the items in the list, all of the items in the list, and any combination of the items in the list.

The above-detailed description of embodiments of the disclosure is not intended to be exhaustive or to limit the teachings to the precise form disclosed above. While specific embodiments of and examples for the disclosure are described above for illustrative purposes, various equivalent modifications are possible within the scope of the disclosure, as those skilled in the relevant art will recognize. For example, while processes or blocks are presented in a given order, alternative embodiments may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, or may be performed, at different times. Further any specific numbers noted herein are only examples: alternative implementations may employ differing values or ranges.

The teachings of the disclosure provided herein can be applied to other systems, not necessarily the system described above. The elements and acts of the various embodiments described above can be combined to provide further embodiments.

Any patents and applications and other references noted above, including any that may be listed in accompanying filing papers, are incorporated herein by reference in their entirety. Aspects of the disclosure can be modified, if necessary, to employ the systems, functions, and concepts of the various references described above to provide yet further embodiments of the disclosure.

These and other changes can be made to the disclosure in light of the above

Detailed Description of the Preferred Embodiments. While the above description describes certain embodiments of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features or aspects of the disclosure with which that terminology is associated. In general, the terms used in the following claims should not be construed to limit the disclosures to the specific embodiments disclosed in the specification unless the above Detailed Description of the Preferred Embodiments section explicitly defines such terms. Accordingly, the actual scope of the disclosure encompasses not only the disclosed embodiments, but also all equivalent ways of practicing or implementing the disclosure under the claims.

Accordingly, although exemplary embodiments of the invention have been shown and described, it is to be understood that all the terms used herein are descriptive rather than limiting, and that many changes, modifications, and substitutions may be made by one having ordinary skill in the art without departing from the spirit and scope of the invention.

What is claimed is:

1. A portable body fluid collection device comprising:
an apparatus for controlling the collection of body fluids that includes
a drainage tube, and
a fluid collection chamber in fluid communication with the drainage tube, wherein the fluid collection chamber includes a first valve, and wherein the fluid collection chamber is configured such that when a predetermined amount of fluid is collected in the collection chamber before a first predetermined period of time elapses, the collection chamber ceases collecting fluid, and
at least one strap associated with the apparatus for controlling the collection of body fluids, wherein the strap can be worn by a patient to support the weight of the automated body fluid drain control apparatus.

2. The portable body fluid collection device of claim 1 wherein the apparatus for controlling the collection of body fluids further includes a measuring device that measures the amount of fluid entering the collection chamber through the first valve during the predetermined first period of time, wherein the first valve has an open and a closed state, wherein the first valve is normally open and allows body fluids to flow into the collection chamber, wherein if during the first period of time a predetermined volume of body fluid enters the chamber, the first valve is closed.

3. The portable body fluid collection device of claim 2 wherein the apparatus for controlling the collection of body fluids further includes a second valve having an open and a closed state, wherein the second valve is normally closed so that body fluids collect in the chamber, and wherein the second valve can be opened to empty the collection chamber.

4. The portable body fluid collection device of claim 3 wherein after the first period of time has elapsed, the first valve is opened and the second valve is closed and a second period of time starts, wherein the first period of time is equal to the second period of time.

5. The portable body fluid collection device of claim 1 further comprising a container, wherein the apparatus for controlling the collection of body fluids is disposed in the container, and the strap is connected to the container.

6. The portable body fluid collection device of claim 5 wherein the drainage tube is configured to be inserted into a patient's subarachnoid region at an insertion point, and wherein the container and strap are configured such that when the container is worn by a patient, the first valve is positioned below the insertion point.

7. The portable body fluid collection device of claim 6 wherein the container is vertically adjustable between at least a first and a second position.

8. The portable body fluid collection device of claim 7 wherein the container is a backpack.

9. A method of collecting body fluid, the method comprising the steps of:
providing an apparatus for controlling the collection of body fluids that includes
a drainage tube, and a fluid collection chamber in fluid communication with the drainage tube, wherein the fluid collection chamber includes a first valve, and wherein the fluid collection chamber is configured such that when a predetermined amount of fluid is collected in the collection chamber before a first predetermined period of time elapses, the collection chamber ceases collecting fluid,
inserting an end of the drainage tube into a patient's subarachnoid region at an insertion point,
securing the apparatus for controlling the collection of body fluids to the patient's body at a first position, wherein in the first position, the first valve is located vertically below the insertion point.

10. The method of claim 9 wherein the apparatus for controlling the collection of body fluids is secured to the patient's body via a strap.

11. The method of claim 10 wherein the strap is connected to a container, and wherein at least a portion of the apparatus for controlling the collection of body fluids is disposed in the container.

12. The method of claim 11 wherein the container is a backpack.
